(19) United States
(12) Patent Application Publication
(10) Pub. No.: US 2023/0233457 A1
(43) Pub. Date: Jul. 27, 2023

(54) PHARMACEUTICAL COMPOSITION CONTAINING GLUTAMINE, PREPARATION AND USE THEREOF

(71) Applicant: MoleculeX Co., Ltd., Hsinchu City (TW)

(72) Inventors: Shih-Yung Liao, Hsinchu City (TW); Yi-Shan Lu, Hsinchu City (TW)

(73) Assignee: MoleculeX Co., Ltd., Hsinchu City (TW)

(21) Appl. No.: 18/156,390

(22) Filed: Jan. 19, 2023

Related U.S. Application Data
(60) Provisional application No. 63/302,138, filed on Jan. 24, 2022.

(51) Int. Cl. A61K 9/00; A61K 31/198

(52) U.S. Cl. CPC A61K 9/006; A61K 31/198

(57) ABSTRACT

A pharmaceutical composition, a preparation and a use for treating oral mucosal wounds. The pharmaceutical composition includes a glutamine, s mucoadhesive polymer, and a slow-release polymer. The mucoadhesive polymer is charged polymer. The slow-release polymer is uncharged polymer. Based on the total weight of the pharmaceutical composition, the content of glutamine is 5% by weight to 75% by weight, the content of mucoadhesive polymer is 20% by weight to 70% by weight, and the content of slow-release polymer is 20% by weight to 70% by weight.

### CROSS-REFERENCE TO RELATED APPLICATION

This application claims the priority benefit of U.S. provisional application Ser. No. 63/302,138, filed on Jan. 24, 2022. The entirety of the above-mentioned patent application is hereby incorporated by reference herein and made a part of this specification.

### BACKGROUND

### Technical Field

The present disclosure relates to a pharmaceutical composition, and in particular to a pharmaceutical composition containing glutamine, a preparation and a use thereof.

### Description of Related Art

Glutamine is a substance used by patients with long-term oral mucositis, which can promote the healing of mucosal wounds and prevent mucosal inflammation or ulcers. Glutamine is currently administered in oral formulations. However, after being absorbed into the hepatic portal vein system through the digestive tract, the dose of glutamine that can enter the circulation in the body will be reduced due to the first pass effect; furthermore, the volume ratio of the oral mucosa in the systemic system is extremely low, so only a small percentage of glutamine can reach the oral mucosa and be absorbed by the mucosal tissues. In addition, some studies have found that glutamine may be absorbed by tumor cells and increase the growth rate of tumor cells, resulting in increased risk of cancer or accelerated cancer progression. Therefore, a large amount of glutamine enters the circulation in the body, resulting in concerns about health and safety.

Based on the low efficiency of the existing use routes of glutamine and the safety concerns of high-dose use, of glutamine it is necessary to provide a precise delivery, low-dose, and high-efficiency of use method.

### SUMMARY

The present disclosure provides a pharmaceutical composition containing glutamine, a preparation and a use thereof, which can be used to treat oral mucosal wounds, and have the advantages of precise delivery to the affected area, low dose and high efficiency of use.

The pharmaceutical composition for treating oral mucosal wounds of the present disclosure includes a glutamine, a mucoadhesive polymer and a slow-release polymer. The mucoadhesive polymer is a charged polymer. The slow-release polymer is an uncharged polymer. Based on the total weight of the pharmaceutical composition, the content of the glutamine is 5% by weight to 75% by weight, the content of the mucoadhesive polymer is 20% by weight to 70% by weight, and the content of the slow-release polymer is 20% by weight to 70% by weight.

In an embodiment of the present disclosure, the above-mentioned mucoadhesive polymer includes an artificially synthesized polymer, a protein, a polysaccharide or a combination thereof, and has a negatively charged group.

In an embodiment of the present disclosure, the above-mentioned electronegative group includes a carboxyl group and a sulfate group.

In an embodiment of the present disclosure, the above-mentioned mucoadhesive polymer is a branched and edible polysaccharide.

In an embodiment of the present disclosure, the above-mentioned mucoadhesive polymer includes carrageenan, pectin, sodium carboxymethyl starch, xanthan gum, gelatin, hyaluronic acid or a combination thereof.

In an embodiment of the present disclosure, the above-mentioned slow-release polymer includes an artificially synthesized polymer, a protein, a polysaccharide or a combination thereof.

In an embodiment of the present disclosure, the viscosity of the 2% aqueous solution of the above-mentioned slow-release polymer is 1000 cP to 200000 cP.

In an embodiment of the present disclosure, the above-mentioned slow-release polymer includes methylcellulose, hydroxyethylcellulose, guanhua bean gum, glucomannan, locust bean gum, hydroxypropyl methylcellulose, wheat protein, soybean protein, whey protein or a combination thereof.

In an embodiment of the present disclosure, based on the total weight of the above-mentioned pharmaceutical composition, the content of the glutamine is 5% by weight to 50% by weight, the content of the mucoadhesive polymer is 25% by weight to 65% by weight, and the content of the slow-release polymer is 25% by weight to 65% by weight.

The use of the pharmaceutical composition of the present disclosure for preparing medicines for treating oral mucosal wounds, wherein the pharmaceutical composition includes a glutamine, a mucoadhesive polymer and a slow-release polymer. The mucoadhesive polymer is a charged polymer. The slow-release polymer is an uncharged polymer. Based on the total weight of the pharmaceutical composition, the content of the glutamine is 5% by weight to 75% by weight, the content of the mucoadhesive polymer is 20% by weight to 70% by weight, and the content of the slow-release polymer is 20% by weight to 70% by weight.

The preparation of the present disclosure of preparing medicines for treating oral mucosal wounds. The preparation includes the above-mentioned pharmaceutical composition.

In an embodiment of the present disclosure, the dosage form of the above-mentioned preparation includes patch, film, lozenge or colloid.

Based on the above, through including the specific ratio of glutamine, mucoadhesive polymer and slow-release polymer, the pharmaceutical composition of the present disclosure can stay on the surface of the oral mucosa (that is, the mucosal wounds) for long-acting, and can last for a long time to release glutamine in the local area, such that glutamine is directly absorbed by mucosal tissues, and thereby increasing the dose absorbed by the local mucosa and reducing the total dose entering the body, achieving the therapeutic effect of promoting mucosal wound healing, and having the advantages of precise delivery to the affected area, low dose and high efficiency of use.

### DESCRIPTION OF THE EMBODIMENTS

Hereinafter, embodiments of the present disclosure will be described in detail. However, these embodiments are illustrative, and the present disclosure is not limited thereto.

Herein, a range represented by being from a value to another value is a schematic representative manner of preventing all values within the range from being listed one by one in the specification. Therefore, a record of a particular value range covers any value within the value range and a smaller value range defined by any value within the value range, like a case in which the any value and the smaller value range are explicitly written in the specification.

In the present disclosure, the pharmaceutical composition includes a glutamine, a mucoadhesive polymer and a slow-release polymer. The glutamine, as the main active ingredient, promotes the repair of oral mucosal wounds and prevent oral mucosal inflammation or ulcers. The mucoadhesive polymer and the slow-release polymer act as long-acting carriers together for active ingredients, and the mucoadhesive polymer can allow glutamine to be precisely attached to specific areas of the mucosa (for example, wounds of oral mucosa), while the slow-release polymer can control the long-acting release of glutamine. In the present disclosure, by adjusting the glutamine, the mucoadhesive polymer and the slow-release polymer into a pharmaceutical composition with a specific ratio, the pharmaceutical composition can stay on the local surface of the oral mucosa for long-acting, and last for a long time to release glutamine in the local area, so that glutamine can be directly absorbed by mucosal tissues, thereby increasing the dose absorbed by local mucosa and reducing the total dose entering the body. Therefore, the pharmaceutical composition of the present disclosure can be precisely positioned at a specific site in the oral mucosa, and has the advantages of low dose of active ingredients and high efficiency of use.

In addition, the pharmaceutical composition containing glutamine may be formulated in to the preparation. In some embodiments, the dosage form of the preparation may include (but not limited to) patch, film, lozenge or colloid. By being made into different dosage forms, the application of the pharmaceutical composition of the present disclosure can be expanded (for example, application in other parts of the body). Hereinafter, each ingredient in the pharmaceutical composition of the present disclosure will be described in detail.

### Glutamine

The glutamine, as the main active ingredient, is used to promote the healing of oral mucosal wounds and prevent oral mucosal inflammation or ulcers. In addition, in the pharmaceutical composition of the present disclosure, other secondary active ingredients may be selectively added to act together with glutamine to accelerate the recovery of mucosal wounds or strengthen the prevention of mucosal inflammation and ulcers.

In some embodiments, based on the total weight of the pharmaceutical composition, the content of the glutamine may be, for example, 5% by weight to 75% by weight. In some other embodiments, based on the total weight of the pharmaceutical composition, the content of the glutamine may be, for example, 5% by weight to 50% by weight. When the content of the glutamine is less than 5% by weight, the content of the glutamine is too low, which may cause the mucosal tissues to fail to absorb a sufficient dose of glutamine, thereby failing to effectively promote the healing of the mucosal wounds. When the content of the glutamine is greater than 75% by weight, the pharmaceutical composition may not be formed, too broken, or unable to withstand a certain degree of bending or breaking in the mouth, resulting in the pharmaceutical composition being unable to effectively stay on the mucosal surface and difficult to handle.

### Mucoadhesive Polymer

The mucoadhesive polymer is used to attach the pharmaceutical composition to the local area, such as oral mucosal wounds, so that active ingredients can be precisely applied to the specific area. The mucoadhesive polymer may be charged polymer, and the molecular weight thereof may be, for example, 5 kilodaltons (kDa) to 5000 kDa. Specifically, the mucoadhesive polymer may, for example, include (but not limited to) artificially synthesized polymer, protein, polysaccharide or a combination thereof. The mucoadhesive polymer may have a negatively charged group, wherein the negatively charged group may include (but not limited to) a carboxyl group and a sulfate group, for example. That is to say, the material of the mucoadhesive polymer may have functional group with divalent or trivalent charges in a neutral environment. In some embodiments, the mucoadhesive polymer may be, for example, a branched and edible polysaccharide, such as carrageenan, pectin, sodium carboxymethyl starch (SSG), xanthan gum, etc., but the present disclosure is not limited thereto. In addition, the protein type of mucoadhesive polymer may include gelatin, hyaluronic acid, etc., but the present disclosure is not limited thereto.

In some embodiments, based on the total weight of the pharmaceutical composition, the content of the mucoadhesive polymer is 20% to 70% by weight. In some other embodiments, based on the total weight of the pharmaceutical composition, the content of the mucoadhesive polymer is 25% to 65% by weight. When the content of the mucoadhesive polymer is less than 20% by weight, the pharmaceutical composition may not be effectively attached to the mucosal tissues due to the low content of the mucoadhesive polymer. When the content of mucoadhesive polymer is greater than 70% by weight, because the mucoadhesive polymer has a faster dissolution rate in water, even if the pharmaceutical composition adheres to the mucous surface, it will still be dissolved and disappeared by the moisture in the environment in a short time, making it impossible to maintain the long-term adhesion of the pharmaceutical composition.

### Slow-Release Polymer

The slow-release polymer, as the active ingredient release carrier, plays the role of slow releasing the active ingredient. The slow-release polymer is an uncharged polymer, and the molecular weight thereof may be, for example, 5 kDa to 5000 kDa. Specifically, the slow-release polymer may include (but not limited to) artificially synthesized polymer, protein, or polysaccharide. In some embodiments, the slow-release polymer may include (but not limited to) methylcellulose, hydroxyethylcellulose, guanhua bean gum, glucomannan, locust bean gum, hydroxypropyl methylcellulose, wheat protein, soybean protein, whey protein or a combination thereof. The slow-release polymer has slow water solubility and may form high-viscosity fluid or gel in water to delay the release time of active ingredients from the carrier. Specifically, the material of the slow-release polymer may form a surface hydration layer after contact with water, which slows down the penetration of surface water into the interior. Usually, the aqueous solution of the slow-release polymer is not just a high-viscosity fluid, but a colloid with solid characteristics. In some embodiments, the viscosity of the aqueous solution containing 2% by weight of the slow-release polymer may be, for example, 1000 cP to 200000 cP.

Since the slow-release polymer has slow water solubility, it can delay the dissolution time of the mucoadhesive polymer. The mixing of mucoadhesive polymer and slow-release polymer involves a compromise between the carrier's mucoadhesive strength and dissolution time. If the proportion of the mucoadhesive polymer is too high, the carrier will be dissolved in a short time, while if the proportion of the slow-release polymer is too high, the carrier will have insufficient mucoadhesive strength, and cannot be effectively fixed on the mucosal surface. In some embodiments, based on the total weight of the pharmaceutical composition, the content of the slow-release polymer is 20% by weight to 70% by weight. In some other embodiments, based on the total weight of the pharmaceutical composition, the content of the slow-release polymer is 25% by weight to 65% by weight.

Hereinafter, the above-mentioned pharmaceutical composition of the present disclosure will be described in detail by means of Experimental Examples. However, the following Experimental Examples are not intended to limit the present disclosure.

### Experimental Method

<Preparation of Pharmaceutical Composition Containing Glutamine (Film Dosage Form)>

In the following examples, the preparation method of the pharmaceutical composition containing glutamine includes but not limited to the following steps.

1. The mucoadhesive polymer (powder), the slow-release polymer (powder), and the glutamine (powder) were mixed in specific proportions to form a powder mixture with a total weight of 4 grams (g). Next, 36 g of pure water and 0.4 g of glycerin were added, and stirred until a homogeneous gel was formed.

2. The homogeneous gel was coated on the release film, and the thickness of the homogeneous gel was about 2 mm. Then, the release film with the homogeneous gel thereon was baked in an oven to form a dried pharmaceutical composition (film dosage form) on the release film. So far, the preparation of the pharmaceutical composition (film dosage form) has been completed.

<90° Peel Adhesion Strength Test>

The pharmaceutical composition (film dosage form) was attached on the porcine oral mucosa fixed on the test platform, and a force (50N) perpendicular to the film was applied at one end of the film by the tension tester, and the film was peeled off at a rate of 3 mm/min until the film was completely peeled off.

Peel strength (N/m)=maximum load (N)/film length (0.008 m).

If the peel strength>80 N/m, it was judged as OO
If 80 N/m>peel strength>20 N/m, it was judged as O
If the peel strength<20 N/m, it was judged as X

<In Vitro Glutamine Release Test>

The pharmaceutical composition loaded with glutamine (film dosage form) was soaked into a phosphate buffered saline (PBS) solution, and the temperature was maintained at 37° C., 50 μL of PBS release solution was drew every 5 minutes, Glutamine/Glutamate-Glo™ Assay (Promega) was used to analyze the concentration of glutamine in the PBS release solution, while 50 μL of fresh PBS solution was replenished at the same time. The half release time (T_{1/2}) was the time point at which 50% of glutamine is released.

If T_{1/2}>2 hours, it was judged as OO
If 2 hours>T_{1/2}>1 hour, it was judged as O
If 1 hour>T_{1/2}, it was judged as X

<Bending Strength Test>

A dried carrier with a length and width of 12.7 mm×125 mm and loaded with or without glutamine was taken and placed in a bending tester (model INSTRON 5565), and then the dried carrier was pressurized from top to bottom at a speed of 15 mm/min at the center of the two fulcrums until the sample broke or exceeded the limit of the testing machine, which is the maximum bending strength FBₘₐₓ (N) of the carrier.

If as exceeding the limit of the testing machine the carrier still does not break (>150N), it was judged as OOO
If FBₘₐₓ>50N, it was judged as OO
If 50N>FBₘₐₓ>15N, it was judged as O
If FBₘₐₓ<15N, it was judged as X

<Cell Scratch Test>

1. Human skin fibroblasts were cultured on a petri dish, a sterilized micropipette was used to scratch straight along the midline of the petri dish, and sterilized saline was used to wash away the scraped cell debris to form a cell-free area with width of about 0.5 mm.

2. After administering different pharmaceutical compositions or glutamine powder, PBS solution was added to rinse for five minutes and then sucked out, and finally the human skin fibroblasts were cultured in groups with glutamine-free cell culture medium.

3. The step 2 was repeated every 4 hours, and the previous culture medium was sucked out, which continued for five days.

Analysis: a microscope was used to observe and take pictures every 24 hours, and the widths of 20 cell-free regions were randomly measured to obtain the average value as Gap_{avg}; when Gap_{avg}<0.1 mm, it was regarded as healing, and the number of experimental days passed by as healing was regarded as T_{recovery}.

When T_{recovery} was less than 120 hours, it was considered to have the effect of promoting healing.

<Evaluation of Wound Protection Effect (Evaluation of Pain Index)>

Subjects with oral wounds rinsed their mouths with drinking water before using the pharmaceutical composition (film dosage form), and a score of 0-10 was proposed based on subjective wound pain perception, with 0 representing no pain at all and 10 representing intolerable pain.

The subjects used the pharmaceutical composition (film dosage form) to cover the wound surface for 10 minutes and then rinsed their mouths with drinking water. According to the subjective wound pain perception, a score of 0-10 was proposed, with 0 representing no pain at all and 10 representing very painful.

The reduction of the pain score after using the pharmaceutical composition determines that the pharmaceutical composition (film dosage form) has a wound protective effect.

### Experimental Example 1. Relative Content of Mucoadhesive Polymer and Slow-Release Polymer

In order to understand the effects of the contents of mucoadhesive polymer and slow-release polymer in the pharmaceutical composition on the mucoadhesive properties and the release time of glutamine (i.e., the slow-release effect), in Experimental Example 1, the content of glutamine in the pharmaceutical composition was fixed at 10% by weight, pectin was used as the mucoadhesive polymer, methylcellulose was used as the slow-release polymer, and the pharmaceutical compositions (film dosage form) of Examples 1 to 9 were prepared according to the composition ratios shown in Table 1. Next, the mucoadhesive strength test and the in vitro glutamine release test were carried out, and the results are shown in Table 2.

TABLE 1

| | mucoadhesive polymer | slow-release polymer | glutamine |
|---|---|---|---|
| | (wt %) | (wt %) | (wt %) |
| Example 1 | 10 | 80 | 10 |
| Example 2 | 20 | 70 | 10 |
| Example 3 | 25 | 65 | 10 |
| Example 4 | 35 | 55 | 10 |
| Example 5 | 45 | 45 | 10 |
| Example 6 | 55 | 35 | 10 |
| Example 7 | 65 | 25 | 10 |
| Example 8 | 70 | 20 | 10 |
| Example 9 | 80 | 10 | 10 |

TABLE 2

| | 90° Peel Adhesion | | In vitro glutamine | |
|---|---|---|---|---|
| | Strength Test | | release test | |
| | peel strength | | T_{1/2} | |
| | (N/cm) | judgment | (minute) | judgment |
| Example 1 | 10 | X | 131 | ◯◯ |
| Example 2 | 18 | X | 113 | ◯◯ |
| Example 3 | 32 | ◯ | 108 | ◯◯ |
| Example 4 | 45 | ◯ | 97 | ◯ |
| Example 5 | 53 | ◯ | 98 | ◯ |
| Example 6 | 78 | ◯ | 95 | ◯ |
| Example 7 | 85 | ◯◯ | 63 | ◯ |
| Example 8 | 89 | ◯◯ | 56 | X |
| Example 9 | 91 | ◯◯ | 32 | X |

It can be seen from Table 2 that when the content of the mucoadhesive polymer gradually increases, the adhesive strength (peel strength) of the pharmaceutical composition will also increase accordingly. When the content of the slow-release polymer gradually increases, the slow-release effect of the pharmaceutical composition will also increase accordingly. Examples 3 to 7 have both mucoadhesive and slow-release effect. In other words, when the content of mucoadhesive polymer per unit of pharmaceutical composition (film dosage form) is between 20% and 70% by weight, and the content of slow-release polymer per unit of pharmaceutical composition (film dosage form) is between 20% and 70% by weight, the pharmaceutical composition can have sufficient mucoadhesive strength and slow-release effect.

### Experimental Example 2. Effect of Glutamine Content on Mechanical Properties

In order to understand the effect of the content of glutamine in the pharmaceutical composition on the mechanical properties of the pharmaceutical composition, in Experimental Example 2, the ratio of the content of the mucoadhesive polymer to the content of the slow-release polymer was fixed at 1:1, and the content of glutamine in the pharmaceutical composition was adjusted for test. In Experimental Example 2, pectin was used as the mucoadhesive polymer, methylcellulose was used as the slow-release polymer, and the pharmaceutical compositions (film dosage forms) of Examples 10-16 were prepared according to the composition ratios shown in Table 3. Next, a bending test was performed to test the effect of the content of glutamine on the mechanical properties of the pharmaceutical composition (film dosage form), and the results are shown in Table 4.

TABLE 3

| | mucoadhesive polymer | slow-release polymer | glutamine |
|---|---|---|---|
| | (wt %) | (wt %) | (wt %) |
| Example 10 | 50 | 50 | 0 |
| Example 11 | 47.5 | 47.5 | 5 |
| Example 12 | 45 | 45 | 10 |
| Example 13 | 38 | 38 | 25 |
| Example 14 | 25 | 25 | 50 |
| Example 15 | 13 | 13 | 75 |
| Example 16 | 5 | 5 | 90 |

TABLE 4

Bending Test

| | FBₘₐₓ (N) | judgment |
|---|---|---|
| Example 10 | >150 | ◯◯◯ |
| Example 11 | >150 | ◯◯◯ |
| Example 12 | >150 | ◯◯◯ |
| Example 13 | 121 | ◯◯ |
| Example 14 | 30 | ◯ |
| Example 15 | 11 | X |
| Example 16 | 9 | X |

It can be seen from Table 4 that in the pharmaceutical composition, when the content of glutamine is greater than or equal to 75% by weight (Examples 15-16), the pharmaceutical composition (film dosage form) will have poor mechanical strength. Therefore, when glutamine accounts for less than 75% by weight in the pharmaceutical composition, it may have better mechanical properties.

### Experimental Example 3. Effect of Glutamine Content on Wound Healing

In order to understand the effect of the content of glutamine in the pharmaceutical composition on wound healing, in Experimental Example 3, the ratio of the content of mucoadhesive polymer to the content of slow-release polymer was fixed at 1:1, and the content of glutamine in the carrier was adjusted for test. In Experimental Example 3, pectin was used as the mucoadhesive polymer, methylcellulose was used as the slow-release polymer, and the pharmaceutical compositions (film dosage forms) of Examples 11 to 14 and 17 were prepared according to the composition ratios shown in Table 5. Next, a cell scratch test was performed to evaluate the effect of the content of glutamine on wound healing, and the results are shown in Table 6.

TABLE 5

| | mucoadhesive polymer | slow-release polymer | glutamine |
|---|---|---|---|
| | (wt %) | (wt %) | (wt %) |
| Example 11 | 47.5 | 47.5 | 5 |
| Example 12 | 45 | 45 | 10 |
| Example 13 | 38 | 38 | 25 |
| Example 14 | 25 | 25 | 50 |
| Example 17 | 0 | 0 | 100 |

TABLE 6

| | Cell Scratch Test | | | | | |
|---|---|---|---|---|---|---|
| | Gap_{avg} (mm) | | | | | |
| | 24 | 48 | 72 | 96 | 120 | T_{recovery} |
| | hours | hours | hours | hours | hours | (hour) |
| Example 11 | 0.41 | 0.32 | 0.16 | 0.12 | 0.09 | 120 |
| Example 12 | 0.33 | 0.26 | 0.15 | 0.09 | 0.06 | 96 |
| Example 13 | 0.28 | 0.16 | 0.08 | 0.06 | 0.00 | 72 |
| Example 14 | 0.24 | 0.17 | 0.06 | 0.05 | 0.00 | 72 |
| Example 17 | 0.44 | 0.37 | 0.30 | 0.25 | 0.19 | >120 |

It can be seen from Table 6 that when the proportion of glutamine in the pharmaceutical composition is lower than or equal to 5% by weight (Example 11), there is a longer cell scratch recovery time, which means that the effect of the pharmaceutical composition on wound healing is lower. Therefore, it is a better design for glutamine to account for more than 5% by weight in the pharmaceutical composition. What is specifically stated here is that in Example 17, because there is no carrier formed by the mucoadhesive polymer and the slow-release polymer, glutamine may not be able to effectively stay (or not be able to stay for a long time) on the cell surface for subsequent being absorbed by the cells. Therefore, compared with Examples 11-16, the cell scratch recovery effect of Example 17 is significantly poor.

### Experimental Example 4. Selection of Mucoadhesive Polymer and Slow-Release Polymer

In order to understand whether different mucoadhesive polymers and different slow-release polymers can lead to the same effect, different materials of mucoadhesive polymer and different materials of slow-release polymer were selected for test. In Experimental Example 4, the pharmaceutical compositions (film dosage forms) of Examples 18-30 were prepared according to the compositions and ratios shown in Table 7. Then, compare whether the results of mucoadhesive strength, in vitro glutamine release and bending test of Examples 18-30 are similar to those of the carrier composed of pectin (as a mucoadhesive polymer) and methylcellulose (as a slow-release polymer), and the results are shown in Table 8.

TABLE 7

| | mucoadhesive polymer | slow-release polymer | glutamine |
|---|---|---|---|
| | 45 wt % | 45 wt % | 10 wt % |
| Example 18 | xanthan gum | hydroxyethylcellulose | glutamine |
| Example 19 | xanthan gum | methylcellulose | glutamine |
| Example 20 | xanthan gum | guanhua bean gum | glutamine |
| Example 21 | sodium carboxymethyl | methylcellulose | glutamine |
| | starch | | |
| Example 22 | sodium carboxymethyl | glucomannan | glutamine |
| | starch | | |
| Example 23 | pectin | locust bean gum | glutamine |
| Example 24 | pectin | glucomannan | glutamine |
| Example 25 | pectin | hydroxypropyl | glutamine |
| | | methylcellulose | |
| Example 26 | xanthan gum | wheat protein | glutamine |
| Example 27 | xanthan gum | poly (methyl | glutamine |
| | | methacrylate | |
| Example 28 | poly(acrylic acid) | hydroxypropyl | glutamine |
| | | methylcellulose | |
| Example 29 | carrageenan | hydroxypropyl | glutamine |
| | | methylcellulose | |
| Example 30 | gelatin | hydroxypropyl | glutamine |
| | | methylcellulose | |

TABLE 8

| | 90° Peel | | | | | |
|---|---|---|---|---|---|---|
| | Adhesion | | In vitro | | | |
| | Strength Test | | glutamine | | Bending | |
| | peel | | release test | | Test | |
| | strength | judg- | T_{1/2} | judg- | FBₘₐₓ | judg- |
| | (N/cm) | ment | (minute) | ment | (N) | ment |
| Example 16 | 81 | ◯◯ | 121 | ◯◯ | 23 | ◯ |
| Example 17 | 56 | ◯ | 13 | ◯◯ | 87 | ◯◯ |
| Example 18 | 85 | ◯◯ | 123 | ◯◯ | 74 | ◯◯ |
| Example 19 | 41 | ◯ | 110 | ◯ | N/A | ◯◯◯ |
| Example 20 | 86 | ◯◯ | 92 | ◯ | N/A | ◯◯◯ |
| Example 21 | 85 | ◯◯ | 136 | ◯◯ | 57 | ◯◯ |
| Example 22 | 51 | ◯ | 92 | ◯ | 23 | ◯ |
| Example 23 | 43 | ◯ | 99 | ◯ | 21 | ◯ |
| Example 26 | 83 | ◯◯ | 126 | ◯◯ | 70 | ◯◯ |
| Example 27 | 47 | ◯ | 126 | ◯◯ | N/A | ◯◯◯ |
| Example 28 | 95 | ◯◯ | 103 | ◯ | 54 | ◯◯ |
| Example 29 | 43 | ◯ | 122 | ◯◯ | 83 | ◯◯ |
| Example 30 | 87 | ◯◯ | 74 | ◯ | 75 | ◯◯ |

It can be seen from Table 7 and Table 8 that the mutual combination between different mucoadhesive polymers and different slow-release polymers may have similar effects.

### Experimental Example 5. Evaluation of Wound Protection Effect (Evaluation of Pain Index)

In order to understand whether by adhering the pharmaceutical composition to the oral mucosal wound, the pharmaceutical composition has a protective effect on the wound and can relieve the pain sensation of the patient, in Experimental Example 5, the pharmaceutical composition (film dosage form) of the aforementioned Example 10 (that is, the content of mucoadhesive polymer is 45% by weight, the content of the slow-release polymer is 45% by weight and the content of glutamine is 10% by weight) is administered to the affected part of the subject, and the wound protection effect is evaluated (evaluation of the pain index), and the results are shown in Table 9.

TABLE 9

| | pain score before | pain score after |
|---|---|---|
| | using the pharmaceutical | using the pharmaceutical |
| | composition (1~10) | composition (1~10) |
| subject 1 | 7 | 2 |
| subject 2 | 5 | 2 |
| subject 3 | 6 | 1 |
| subject 4 | 4 | 1 |
| subject 5 | 7 | 3 |
| subject 6 | 6 | 3 |
| subject 7 | 3 | 1 |
| subject 8 | 7 | 2 |
| subject 9 | 6 | 2 |
| subject 10 | 6 | 3 |
| subject 11 | 4 | 1 |
| subject 12 | 5 | 2 |
| average | 5.5 | 1.9 |

It can be seen from Table 9 that the average pain score before using the pharmaceutical composition was 5.5, and the pain score after using the pharmaceutical composition was 1.9, with an average decrease of 3.6 points, showing that the pharmaceutical composition of the present disclosure has the effect of wound protection and pain relief.

In order to improve the efficiency of glutamine delivery in the oral mucosa, the present disclosure uses a local glutamine carrier, which can adhere to the surface of the oral mucosa and release glutamine locally for a long time, such that glutamine is directly absorbed by mucosal tissues, and thereby increasing the dose absorbed by the local mucosa and reducing the total dose entering the body. The carrier can dissolve slowly and disappear naturally after tens of minutes to several hours without additional removal.

In order to allow the carrier to adhere to the mucous surface, it is necessary to select a polymer material with mucoadhesive properties as the adhesive, and the material usually contains a material with a charged group. However, polymer with mucoadhesive properties usually have a very fast dissolution rate in water. As such, even if the carrier can adhere to the mucous surface, the carrier will still be dissolved and disappeared by the moisture in the environment in a short time, making it not suitable as a long-acting carrier in the oral alone.

In order to solve the problem of easy dissolution of mucoadhesive polymer, a slow-release polymer is added into the composition of the present disclosure. The slow-release polymer usually plays the role of slow releasing pharmaceutical in the pharmaceutical release carrier. The slow-release polymer usually has slow water solubility, and may form high-viscosity fluid or gel in water, so that the release time of pharmaceutical from the carrier is delayed. Therefore, the slow-release polymer can delay the dissolution time of the mucoadhesive polymer.

However, the mixing of mucoadhesive polymer and slow-release polymer involves a compromise between the carrier's mucoadhesive strength and dissolution time. If the proportion of the mucoadhesive polymer is too high, the carrier will be dissolved in a short time, while if the proportion of the slow-release polymer is too high, the carrier will have insufficient mucoadhesive strength, and cannot be effectively fixed on the mucosal surface.

The ratio of glutamine in the carrier will also affect the effectiveness of the method of the present disclosure. If the proportion of glutamine is too low, the mucosa will not to absorb a sufficient dose of glutamine, thereby failing to effectively promote the healing of mucosal wounds. However, if the proportion of glutamine is too high, the carrier cannot be formed, too broken or cannot withstand a certain degree of bending or breaking in the mouth, and then cannot effectively stay on the mucosa surface, and is difficult to handle.

Therefore, the adjustment of the ratio between the mucoadhesive polymer, the slow-release polymer, and glutamine is an important factor of the present disclosure, which directly affects the effectiveness of this usage method for treating oral mucosal wounds.

To sum up, through the combination of the glutamine, the mucoadhesive polymer and the slow-release polymer in a specific ratio, the pharmaceutical composition of the present disclosure can stay on the surface of the oral mucosa (that is, the mucosal wounds) for long-acting, and can continuously release glutamine locally for a long time, so that glutamine can be directly absorbed by the mucosal tissues, and thereby increasing the dose absorbed by the local mucosa and reducing the total dose entering the body, achieving the therapeutic effect of promoting mucosal wound healing, and having the advantages of precise delivery to the affected area, low dose and high efficiency of use.

Although the present disclosure has been disclosed above with experimental examples, it is not intended to limit the present disclosure. Anyone with ordinary knowledge in the technical field may make some changes and modifications without departing from the spirit and scope of the present disclosure. The scope of protection of the present disclosure should be defined by the scope of the appended patent application.

What is claimed is:

1. A pharmaceutical composition for treating oral mucosal wounds, comprising:
glutamine;
a mucoadhesive polymer, being a charged polymer; and
a slow-release polymer, being an uncharged polymer,
wherein based on a total weight of the pharmaceutical composition, a content of the glutamine is 5% by weight to 75% by weight, a content of the mucoadhesive polymer is 20% by weight to 70% by weight, and a content of slow-release polymer is 20% by weight to 70% by weight.

2. The pharmaceutical composition according to claim 1, wherein the mucoadhesive polymer includes an artificially synthesized polymer, a protein, a polysaccharide or a combination thereof, and has a negatively charged group.

3. The pharmaceutical composition according to claim 2, wherein the negatively charged group includes a carboxyl group and a sulfate group.

4. The pharmaceutical composition according to claim 1, wherein the mucoadhesive polymer is a branched and edible polysaccharide.

5. The pharmaceutical composition according to claim 1, wherein the mucoadhesive polymer includes carrageenan, pectin, sodium carboxymethyl starch, xanthan gum, gelatin, hyaluronic acid or a combination thereof.

6. The pharmaceutical composition according to claim 1, wherein the slow-release polymer includes an artificially synthesized polymer, a protein, a polysaccharide or a combination thereof.

7. The pharmaceutical composition according to claim 1, wherein a viscosity of 2% aqueous solution of the slow-release polymer is 1000 cP to 200000 cP.

8. The pharmaceutical composition according to claim 1, wherein the slow-release polymer includes methylcellulose, hydroxyethylcellulose, guanhua bean gum, glucomannan, locust bean gum, hydroxypropyl methylcellulose, wheat protein, soybean protein, whey protein or a combination thereof.

9. The pharmaceutical composition according to claim 1, wherein based on the total weight of the pharmaceutical composition, the content of the glutamine is 5% by weight to 50% by weight, the content of the mucoadhesive polymer is 25% by weight to 65% by weight, and the content of slow-release polymer is 25% by weight to 65% by weight.

10. A use of a pharmaceutical composition for preparing medicines for treating oral mucosal wounds, wherein the pharmaceutical composition comprises:
a glutamine;
a mucoadhesive polymer, being charged polymer; and
a slow-release polymer, being uncharged polymer,
wherein based on a total weight of the pharmaceutical composition, a content of the glutamine is 5% by weight to 75% by weight, a content of the mucoadhesive polymer is 20% by weight to 70% by weight, and a content of slow-release polymer is 20% by weight to 70% by weight.

11. A preparation of preparing medicines for treating oral mucosal wounds, wherein the preparation includes the pharmaceutical composition according to claim 1.

12. The preparation according to claim 11, wherein a dosage form of the preparation comprises patch, film, lozenge or colloid.
